(19) United States
(12) Patent Application Publication
(10) Pub. No.: US 2023/0355515 A1
(43) Pub. Date: Nov. 9, 2023

(54) Substrates for Enhancing Purity or Yield of Compounds Forming a Condensation Aerosol

(71) Applicant: Alexza Pharmaceuticals, Inc., Fremont, CA (US)

(72) Inventors: Krishnamohan SHARMA, Milpitas, CA (US); Mingzu LEI, San Jose, CA (US); Peter D. NOYMER, Los Gatos, CA (US); Daniel J. MYERS, Mountain View, CA (US)

(21) Appl. No.: 18/222,933

(22) Filed: Jul. 17, 2023

Related U.S. Application Data
(62) Division of application No. 16/044,224, filed on Jul. 24, 2018.
Application No. 16/044,224 is a continuation of application No. 12/789,044, filed on May 27, 2010, now abandoned.
(60) Provisional application No. 61/181,867, filed on May 28, 2009.

(51) Int. Cl. A61K 9/00; A61K 31/553; A61M 11/04; B05B 7/00; B05B 7/16; C23C 24/04; A61M 11/00; C23C 4/123

(52) U.S. Cl. CPC A61K 9/007; A61K 31/553; A61M 11/041; B05B 7/0012; B05B 7/1686; C23C 24/04; A61M 11/001; A61M 11/042; A61M 11/047; C23C 4/123; B05D 1/60

(57) ABSTRACT

A device for vaporizing a composition. The device comprises a thermally conductive substrate having a surface, the surface comprising a thermally conductive surface structure. A dry composition capable of a solid to liquid phase change upon being heated to at least a select temperature is disposed on the surface structure. The surface structure is configured to form liquid droplets of the composition upon heating of the surface structure to at least the select temperature. The liquid droplets have a median diameter less than a median diameter of liquid droplets formed on a planar substrate surface heated to at least the select temperature.

### RELATED APPLICATIONS

This application is a divisional of U.S. application Ser. No. 16/044,224, filed Jul. 24, 2018, which is a continuation of U.S. application Ser. No. 12/789,044, filed May 27, 2010. U.S. application Ser. No. 12/789,044 claims priority to U.S. Provisional Application No. 61/181,867, filed on May 28, 2009, the entire disclosures of which are hereby incorporated by reference. Any disclaimer that may have occurred during the prosecution of the above-referenced applications is hereby expressly rescinded, and reconsideration of all relevant art is respectfully requested.

### TECHNICAL FIELD

The present disclosure is directed to devices and methods for producing a condensation aerosol, and more particularly to apparatus and methods for producing a drug condensation aerosol.

### BACKGROUND

Traditionally, inhalation therapy has played a relatively minor role in the administration of therapeutic agents when compared to more traditional drug administration routes of oral delivery and delivery via injection. Due to drawbacks associated with traditional routes of administration, including slow onset, poor patient compliance, inconvenience, and/or discomfort, alternative administration routes have been sought. Pulmonary delivery is one such alternative administration route which can offer several advantages over the more traditional routes. These advantages include rapid onset, the convenience of patient self-administration, the potential for reduced drug side-effects, the ease of delivery by inhalation, the elimination of needles, and the like. Many preclinical and clinical studies with inhaled compounds have demonstrated that efficacy can be achieved both within the lungs and systemically.

However, despite such results, the role of inhalation therapy in the health care field has remained limited mainly to treatment of asthma and other diseases of the respiratory tract, in part due to a set of problems unique to the development of inhalable drug formulations and their delivery modalities, especially formulations for, and delivery by, inhalation.

Metered dose inhaler formulations involve a pressurized propellant, which is frequently a danger to the environment, and generally produces aerosol particle sizes undesirably large for systemic delivery by inhalation. Furthermore, the high speed at which the pressurized particles are released from metered dose inhalers makes the deposition of the particles undesirably dependent on the precise timing and rate of patient inhalation. Also, the metered dose inhaler itself tends to be inefficient because a portion of the dose is lost on the wall of the actuator, and due to the high speed of ejection of the aerosol from the nozzle, much of the drug impacts ballistically on the tongue, mouth, and throat, and never gets to the lungs.

While solving some of the problems with metered dose inhalers, dry powder formulations are prone to aggregation and low flowability phenomena which considerably diminish the efficiency of dry powder-based inhalation therapies. Such problems are particularly severe for dry powders having an aerosol particle size small enough to be optimal for deep lung delivery, as difficulty of particle dispersion increases as particle size decreases. Thus, excipients are needed to produce powders that can be dispersed. This mix of drug and excipient must be maintained in a dry atmosphere lest moisture cause agglomeration of the drug into larger particles. Additionally, it is well known that many dry powders expand as they are delivered to the patient's airways due to the high levels of moisture present in the lung.

Liquid aerosol formations similarly involve non-drug constituents, i.e. the solvent, as well as preservatives to stabilize the drug in the solvent. Thus, all liquid aerosol devices must overcome the problems associated with formulation of the compound into a stable liquid. Liquid formulations must be prepared and stored under aseptic or sterile conditions since they can harbor microorganisms. This necessitates the use of preservatives or unit dose packaging. Additionally, solvents, detergents and other agents are used to stabilize the drug formulation. Moreover, the dispersion of liquids generally involves complex and cumbersome devices and is effective only for solutions with specific physical properties, e.g. viscosity. Such solutions cannot be produced for many drugs due to the solubility properties of the drug.

Recently, devices and methods for generating aerosols via volatilization of the drug have been developed, which addresses many of these above mentioned problems. (See, e.g., Rabinowitz et al., U.S. Publication No. US 2003/0015190, Cross et al., U.S. Publication No. 2005/0268911; Hale et al., U.S. Pat. No. 7,090,830, each incorporated by reference in its entirety). These devices and methods eliminate the need for excipients to improve flowability and prevent aggregation, solvents or propellants to disperse the compound, solution stabilizers, compound solubility, etc. and hence, the associated problems with these added materials. Additionally, devices and methods have been developed that allow for consistent particle size generation using volatilization. With such devices, a drug compound typically is deposited on a surface of a substrate, such as a stainless steel foil. The substrate is rapidly heated to volatilize the drug, followed by cooling of the vapor so that it condenses to form an aerosol (i.e., a condensation aerosol).

Volatilization, however, subjects the drug to potential chemical degradation via thermal, oxidative, catalytic and/or other means. The activation energies of these degradation reactions depend on molecular structure, energy transfer mechanisms, transitory configurations of the reacting molecular complexes, and the effects of neighboring molecules. One method to help control degradation during volatilization is the use of the flow of gas across the surface of the compound, to create a situation in which a compound's vapor molecules are swept away from its surface. (See e.g., Wensley et al., U.S. Publication No. US 2003/0062042 A1). Additionally, the use of thin films reduces the amount of thermal degradation by decreasing the temporal duration of close contact between the heated drug molecule and other molecules and/or the surface on which the drug is in contact. However, these methods have proven inadequate to allow for volatilization and delivery of condensation aerosols of more temperature sensitive drugs. For example, in order to provide rapid vaporization of drug compositions before they are subject to unacceptable degradation, relatively high heats on the order of 400° C. must be applied to the compositions. Certain drug compositions such as Viagra® distributed by Pfizer, Inc. simply cannot be exposed to such high temperatures even when applied as a thin film to a substrate and air flow is provided in communication with the substrate. Other drug compositions require delivery of an amount of the drug per dose which is greater than that which can be effectively deposited as a thin film. In addition, use of the thin film requires a relatively large surface area for the substrate, which increases the size requirement for a drug delivery device. This not only increases material, packaging and transportation costs, but increases the amount of material that must be disposed of after use of the drug delivery device. Moreover, depositing thin layers of compositions on the substrates has proven time consuming. Furthermore, the need to rapidly heat the substrate to a high temperature requires a relatively high energy input which may make heating the substrate by some means, such as resistive heating, impractical. Finally, the high temperatures necessary for rapid volatilization give rise to user safety concerns.

The various embodiments disclosed or claimed herein are directed toward overcoming one or more of the problems discussed above.

### SUMMARY

A first aspect of the invention is a device for vaporizing a composition. The device comprises a thermally conductive substrate having a surface, the surface comprising a thermally conductive surface structure. A dry composition capable of a solid to liquid phase change upon being heated to at least a select temperature is disposed on the surface structure. The surface structure is configured to form liquid droplets of the composition upon heating of the surface structure to at least the select temperature. The liquid droplets have a median diameter less than a median diameter of liquid droplets formed on a planar substrate surface heated to at least the select temperature. In one embodiment the surface structure is three dimensional. The three dimensional surface structure may comprise metallic particles disposed on the first surface of the substrate. The metallic particles may be supported in an inorganic binder, for example, silicate, phosphate or aluminate binders. The metallic particles may have a diameter of less than 45 microns. In each embodiment including the metallic particles, the metallic particles may be made of stainless steel. In one embodiment the substrate comprises a metal foil. The metal foil may be made of steel. In another embodiment the three dimensional surface structure comprises sintered metallic particles. In one embodiment the first surface and the substrate comprise a three dimensional structure and the three dimensional structure comprises metallic particles supported in a silicate binder. In other embodiments, the three dimensional structure may be formed by chemical or laser etching, cladding or other techniques.

A second aspect of the invention is a drug supply unit configured to heat a drug composition coated on a surface of a thermally conductive substrate to a temperature sufficient to vaporize the drug composition. The drug supply unit comprises a surface structure operatively associated with the surface, the surface structure being configured such that a drug composition vaporized from the surface comprising the surface structure exhibits a purity or yield that is greater than the purity or yield of a drug vaporized from the surface without the surface structure for a given drug coating density. In one embodiment the surface structure is three dimensional. The three dimensional surface structure may comprise thermally conductive or metallic particles disposed on the surface. The thermally conductive particles may be suspended in an inorganic binder or may be sintered.

Yet another aspect of the invention is an aerosol drug delivery device comprising a housing defining an airway. A drug supply unit that is configured to heat a drug composition disposed on an exterior surface of a thermally conductive substrate to a temperature sufficient to vaporize the drug composition is disposed in the airway. The exterior surface of the thermally conductive substrate comprises a thermally conductive surface structure. A drug composition disposed on the thermally conductive surface structure. The thermally conductive surface structure is configured to form liquid droplets of the drug composition prior to volatilization of the drug composition having a median diameter less than a median diameter of liquid droplets formed on the surface of the substrate without the thermally conductive surface structure. The surface structure may be three dimensional.

Yet another aspect of the invention is a substrate having a surface for bearing a composition to be vaporized by heating of the substrate. The surface comprises thermally conductive particles of a size less than 45 μm supported in a silicate binder in a manner preventing formation of droplets of the composition on the surface of the substrate of a size greater than 2000 μm in diameter during vaporization of a layer of a composition applied to the substrate.

### BRIEF DESCRIPTION OF THE DRAWINGS

FIG. 1 is a schematic depiction of an aerosol drug delivery device comprising a surface structure for receiving a composition disposed on a surface of a substrate;

FIG. 2 is an enlarged schematic cross-section of a surface structure disposed on a surface of a substrate depicted in FIG. 1;

FIG. 3 is a graph of measured surface temperature versus voltage applied to a planar substrate, a substrate having a surface structure as depicted in FIGS. 1 and 2 disposed thereon of a first thickness and a substrate having the surface structure having a second thickness approximately twice that of the first thickness;

FIG. 4 is a chart depicting temperatures versus emitted dose correlation for a planar stainless steel substrate and substrates having a surface structure as disclosed herein;

FIG. 5 is a series of photographs showing the formation of droplets of a composition on a planar stainless steel substrate as a function of time; and

FIG. 6 is a series of photographs of a surface structure on a substrate coated with the same amount of a composition as the substrate depicted in FIG. 5 demonstrating that no visible droplets form over time.

### DETAILED DESCRIPTION

Unless otherwise indicated, all numbers expressing quantities of ingredients, dimensions reaction conditions and so forth used in the specification and claims are to be understood as being modified in all instances by the term “about”.

In this application and the claims, the use of the singular includes the plural unless specifically stated otherwise. In addition, use of “or” means “and/or” unless stated otherwise. Moreover, the use of the term “including”, as well as other forms, such as “includes” and “included”, is not limiting. Also, terms such as “element” or “component” encompass both elements and components comprising one unit and elements and components that comprise more than one unit unless specifically stated otherwise.

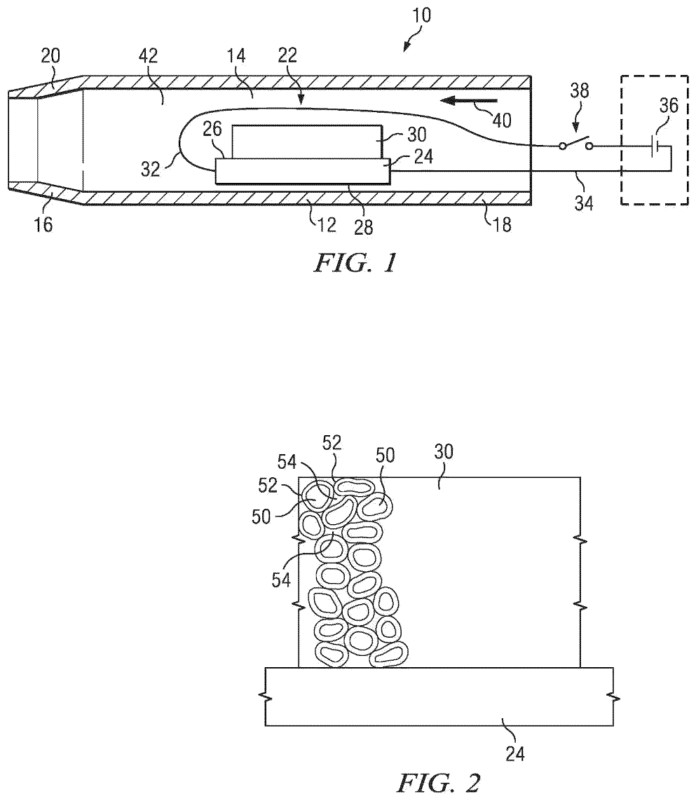

FIG. 1 is a schematic representation of a condensation aerosol drug delivery device 10. The condensation aerosol drug delivery device 10 comprises a housing 12 defining an airway 14 extending between a proximal end 16 and a distal end 18 of the housing 12. A mouthpiece 20 is provided at the proximal end 16. A drug supply unit 22 is disposed in the airway 14. The drug supply unit 22 comprises a thermally conductive substrate 24 having a first surface 26 and a second surface 28. The first surface 24 comprises a thermally conductive surface structure 30. In this embodiment, the surface structure is three dimensional. A dry composition capable of a solid to liquid phase change upon being heated to at least a select temperature is disposed on the thermally conductive surface structure 30. The dry composition may comprise a drug. Electrical leads 32, 34 are connected to opposite sides of the thermally conductive substrate 24 and extend to a power source 36. A switch 38 is provided in electrical communication with one of the leads 32, 34 to allow selective application of electrical current to the substrate 24 by a user.

In use, upon actuation of the switch 38 by a user, electrical current is applied to the substrate 24, heating the substrate 24 and the thermally conductive surface structure 30 to volatilize the composition disposed on the thermally conductive surface structure 30. Simultaneously, a patient inhales using the mouth piece 20 to draw air through the airway 14 in the direction of the arrow 40. The vaporized composition moves from the heated substrate and condenses to form a condensation aerosol in the condensation region 42 of the airway 40. The airflow traveling from the distal end 18 to the mouth piece 20 is inhaled by the user. The user may be human or other mammal.

The specific embodiment of FIG. 1 depicts a condensation aerosol drug delivery device for use by humans or other mammals. The device could be modified to simply produce a condensation aerosol of compositions other than drugs or could be modified to not require inhalation. For example, a supply of pressured gas could be coupled to the distal end to form a condensation aerosol upon vaporization of the composition and the condensation aerosol could be delivered from an outlet at the distal end 16.

FIG. 2 is a schematic representation of the thermally conductive substrate 24 with the first surface 26 comprising a thermally conductive surface structure 30 as depicted in FIG. 1. The thermally conductive surface structure 30 comprises a large number of thermally conductive particles 50 supported in an inorganic binder 52 that effectively surround the thermally conductive particles 50. A interconnected web of air channels 54 are defined between the thermally conductive particles encased in the binder.

The embodiment illustrated in FIG. 2, the thermally conductive substrate 24 may be a metal, metal oxide, thermally conductive ceramic or an organic-inorganic hybdrid. A stainless steel foil having a thickness in a range of 50-200 μm is one example.

The thermally conductive particles 50 may be made of metal, metal oxides, mixed metal oxides, carbides, nitrides, and carbon. In one embodiment, the particles have a diameter of less than 100 μm. In another embodiment, the diameter of the particles is less than 45 μm. In an embodiment found to yield effective results, the particles are stainless steel and the binder 52 is a silicate binder. An example of an acceptable silicate binder is lithium, sodium or potassium silicate or combinations thereof.

The thermally conductive surface structure 30 described above has been found effective for decreasing the size of droplets formed from a composition deposited thereon relative to a planar stainless steel structure as the composition undergoes a solid to liquid phase change during heating and vaporization. In addition to the three dimensional structure illustrated in FIGS. 1 and 2 and described above, other thermally conductive surface structures 30 may be effective for decreasing the size and droplets formed from a dry composition coated on the surface structure during a solid to liquid phase change upon being heated to at least a select temperature for the purpose of vaporizing the dry composition. For example, certain chemicals or compounds could be coated on the first surface 26 of the substrate 24. In addition, various treatments may be applied to the surface 26 of the substrate 24 to yield an acceptable surface structure. For example, the first surface 26 may be chemically etched, plasma etched, cladded, coated via vapor deposition and any other form of surface treatment yielding a thermally conductive surface structure promoting formation of droplets smaller than those resulting from an untreated first surface 26 of the substrate 24.

In some embodiments, the surface structure may be integrally formed with the substrate 24. This may be accomplished by one of the surface treatments discussed above, or could result from the substrate being porous when formed. For example, a substrate formed of sintered metal particles. Such a completely porous substrate could enable an embodiment where air is flowed through the substrate to more quickly remove vaporized composition from the substrate, which could help minimize drug decomposition and increase the speed of vaporization.

In embodiments where the surface structure is a three dimensional surface structure applied to the surface of a substrate as depicted in FIGS. 1 and 2, the three dimensional surface structure may have a thickness ranging from a few microns to over 1000 μm. Embodiments where the thermally conductive three dimensional surface structure has a thickness of 10-2000 μm, have yielded acceptable results. Thicknesses in the range of 100-1000 μm have also proven acceptable.

In the broadest sense, any surface structure configured to form liquid droplets of a composition upon heating of the surface structure to at least a select temperature changing a dry composition deposited thereon from a solid to a liquid phase which produces liquid droplets having a median diameter of liquid droplets smaller than those formed on a planar substrate surface not having the surface structure is within the scope of the invention.

In constructing an aerosol drug delivery device as described herein, a liquid formulation of a drug composition intended to be vaporized is deposited onto the thermally conductive surface structure 30 and then is allowed to dry to leave a dry composition capable of solid to liquid phase change upon being heated to at least a select temperature disposed on the thermally conductive surface structure 30.

The droplet size is a function not only of the surface structure and an enhanced surface area, but is also a function of surface wetting behavior as between a composition in its liquid form and the material forming the surface structure. Hydrophilic materials will tend to promote formation of finer droplets.

The composition may be deposited onto the thermally conductive surface structure using a number of different methods. Such methods include, without limitation, adding a solution of a drug in a volatile organic solvent to the substrate and allowing the solvent to evaporate; dipping the substrate into a solution of a drug in a volatile organic solvent, removing it and allowing the solvent to evaporate; depositing the composition through chemical vapor deposition.

The embodiment illustrated in FIG. 1 provides for heating of the substrate by placing electrodes at either end and passing an electric current through it (i.e., restive heating). Alternatively, isothermal chemical reactants may be applied to the second side of the substrate to heat the substrate. In other embodiments, the substrate can be bonded to a second substrate that is heated. Heating then occurs through thermal conductivity pathways. Non-limiting examples by which the second substrate can be heated include the following: passing current though an electrical resistant element; absorption of electromagnetic radiation, such as microwave or laser light; and exothermic chemical reactions, such as exothermic salvation, hydration of pyrophoric materials and oxidation of combustible materials.

For drug based compositions, the composition is generally heated in one of two forms: as a drug; or as a mixture of a pure drug and a pharmacologically acceptable excipient. Pharmacologically acceptable excipients are either volatile or non-volatile. Volatile excipients, when heated, are concurrently volatilized and inhaled with the drug.

Classes of such excipients are known in the art and include, without limitation, gaseous, supercritical fluid, liquid and solid solvents. The following is a list of nonlimiting examples of carriers within these classes: water; terpenes, such as menthol; alcohols, such as ethanol, propylene glycol, glycerol and other similar alcohols; dimethylformamide; dimethylacetamide; wax; supercritical carbon dioxide; dry ice; and mixtures thereof.

Nonlimiting examples of drugs that may be delivered from a substrate with a surface structure as described herein for use in inhalation therapy include the following: acebutolol, acetaminophen, albuterol, alfenatil, alprazolam, amantadine, amitriptyline, amobarbital, amoxipine, apomorphine diacetate, apomorphine hydrochloride, aripiprazole, aspirin, astemizole, atenolol, atropine, azatidine, baclofen, benazepril, benztropine mesylate, bergapten, beta estradiol, betahistine, biperiden, bromazepam, bromocryptine, brompheniramine, bumetanide, buprenorphine, bupropion, buspirone, butalbital, butorphanol, caffeine, carbamazepine, carbidopa, carbinoxamine maleate, carisoprodol, celecoxib, cetirizine, chloral hydrate, chlordiazepoxide, chlorpheniramine, chlorpromazine, chlorzoxazone, ciclesonider, cinnarizine, citalopram, clemastine, clofazimine, clomipramine, clonazepam, clonidine, clorazepate, clozapine, codeine, colchicine, cyclobenzaprine, cyproheptadine, dapsone, desipramine, dextroamphetamine, dezocine, diazepam, diclofenac ethyl ester, diclofenac, diflunisal, dihydroergotamine, diltiazem, dimenhydrinate, diphenhydramine, dipyridamole, disopyramide, disulfiram, dolasetron, donepezil, doxepin, doxylamine, dronabinol, droperidol, efavirenz, eletriptan, entacapone, ephedrine, ergotamine, escitalopram, esmolol, estazolam, estradiol 17-enanthate, ethacrynic acid, ethosuximide, etodolac, felbamate, fenfluramine, fenoprofen, fentanyl, flecainide, fluconazole, flunisolide, flunitrazepam, fluoxetine, fluphenazine, flurazepam, fluribiprofen, fluticasone proprionate, fluvoxamine, fosphenytoin, frovatriptan, gabapentin, galanthamine, granisetron, haloperidol, hydrocodone, hydromorphone, hydroxychloroquine, hydroxyzine, hyoscyamine, ibuprofen, ibutilide, imipramine, indomethacin, isocarboxazid, isotretinoin, ketamine, ketoprofen ethyl ester, ketoprofen, ketorolac ethyl ester, ketorolac methyl ester, ketorolac, ketotifen, lamotrigine, levetiracetam, levodopa, levorphenol, lidocaine, linezolid, lithium, loperamide, loratadine, lorazepam, lovastatin, loxapine, maprotiline, meclizine, meclofenamate, melatonin, meloxicam, memantine, meperidine, mephobarbital, meprobamate, mesoridazine, metaproterenol, metaxalone, methadone, methocarbamol, methoxsalen, methsuximide, methylphenidate, methylprednisolone, methysergide, metoclopramide, metoclopramide, metoprolol, mexiletine HCl, midazolam, mirtazapine, modafinil, molindone, morphine, nabumetone, nalbuphine, nalmefene, naloxone, naltrexone, naproxen, naratriptan, nefazodone, nicotine, nortriptyline, O-diacetyl, olanzapine, ondansetron, orphenadrine, oxaprozin, oxazepam, oxcarbazepine, oxybutynin, oxycodone, oxymorphone, paracoxib, paroxetine, pemoline, pentazocine, pentobarbital, pergolide, perphenazine, phenelzine, phenobarbital, phentermine, phenytoin, pimozide, pindolol, pioglitazone, piribedil, piroxicam, pramipexole, pregnanalone, primidone, procainamide, prochlorperazine, proeblorperazine, promazine, promethazine, propafenone, propoxyphene, propranolol, protriptyline, pyrilamine, quetiapine, quinine, rauwolfia, remifentanil, risperidone, rizatriptan, rofecoxib, ropinirole, salsalate, scopolamine, secobarbital, selegiline, sertraline, sibutramine, sildenafil, sotalol, spironolactone, sufentanil, sulindac, sumatriptan, tacrine, tadalafil, tamoxifen, telmisartan,.temazepam, terbutaline, testosterone, thalidomide, thambutol, theophylline, thioridazine, thiothixene, tiagabine, tizanidine, tocainide, tolcapone, tolfenamic acid, tolmetin, tolterodine, topiramate, toremifene, tramadol, tranylcypromine, trazodone, triamcinolene acetonide, triamterene, triazolam, trichlormethiazide, trifluoperazine, trihexyphenidyl, trimethobenzamide, trimipramine, valdecoxib, valproic acid, vardenafil, venlafaxine, verapamil, vitamin E, zaleplon, zolmitriptan, zolpidem, zonisamide, zopiclone and zotepine.

One distinct advantage of the use of the three dimensional surface structure is the high surface area of the three dimensional surface structure enables application of a greater volume of a composition for a given footprint of the three dimensional surface structure than could be applied to a planar substrate surface, such as the first surface 26 of the substrate 24. In other words, a larger dose of a drug can be administered for a given footprint surface area of a substrate utilizing a three dimensional surface structure than a substrate not having a three dimensional surface structure. This enables decreasing the required footprint of the substrate required for a given dose of a drug. This has numerous benefits, including decreasing the amount of material required to produce the drug supply unit 22 and the overall size of the drug delivery device 10. Reduction in surface area requirements enables design of a multi-dose device (disposable or reusable) or enables the delivery of drugs that require unusually high surface area for purity or higher dose related reasons. Further, reduction in surface area not only decreases the cost, but minimizes the environmental impact in diminishing the volume of waste generated by the use of delivery devices utilizing a three dimensional surface structure on a substrate. An additional benefit of the three dimensional surface substrate is that it simplifies application of the composition as compared to a conventional planar substrate. This is because conventional planar substrates require a very thin drug coating to provide rapid volatilization and applying a thin, even drug layer is difficult. With the three dimensional surface structure, the liquid formulation can essentially be poured on with minimal precision required. Yet a further advantage is the three dimensional surface structure provides greater physical stability to the substrate structure and the drug solutions could be metered onto the surfaces precisely (which dramatically simplifies the manufacturing of dose cartridges by conforming to FDA's Process Analytical Technology initiative).

Further, it has been found that use of the described surface structures which result in smaller droplets decreases the required temperature to rapidly volatilize the dry composition. This has a particular advantage where the dry composition is a drug that is susceptible to thermal degradation during vaporization. In addition, higher yields and purer drugs are believed to result from formation of smaller droplets during the volatilization stage of at least some drugs. Operating at lower temperatures also improves safety of the device.

A further advantage of the surface structure, depicted in FIGS. 1 and 2, is more efficient heating of the composition coating. Without being bound by theory, the more efficient heating is believed to result from radiative and convective heating within the three dimensional surface structure supplementing the conductive heating by the substrate itself. These factors are believed to speed volatilization of the composition and decrease the amount of energy and time of heating necessary to form the composition vapor.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims. All references cited herein are incorporated in their entirety by reference.

### EXAMPLES

The following example is provided for illustrative purposes only and is not intended to limit the scope of the invention.

### Example 1

9 grams of stainless steel particles having a diameter of less than 45 μm (325 mesh) were suspended in a sodium silicate binder (10 ml, 15% solid) to form a slurry and coated on top of a 127 μm (0.005 inch) thick stainless steel foil (304 full hard) manually. A relatively thin coatings of 9 ml/cm² were made by spreading 25 ml of slurry in an approximately 2.8 cm² surface area while thicker coatings (18 ml/cm²) were made by spreading 50 ml of the slurry on a 2.8 cm² surface area. The coatings were cured at 350° C. for 2 hours to rigidly bind the stainless steel particles to the foil. The specific silicate binder used in this example is sodium silicate (already mentioned above).

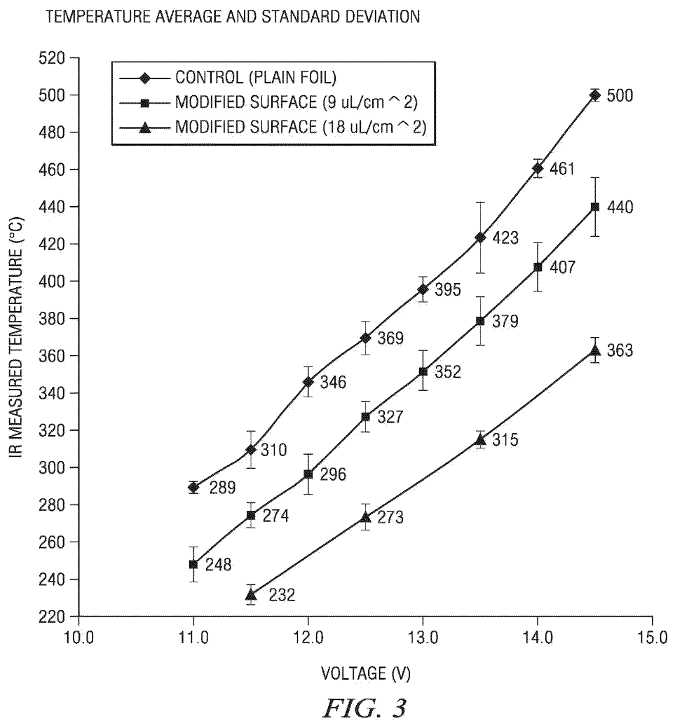

The surface temperature of the steel foils were measured using an infrared camera at different voltages applied using a one Farad capacitor. FIG. 3 depicts the surface temperature as a function of the applied voltage of a plain foil, foil having a 9 ml/cm² surface coating and a substrate having 18 ml/cm² surface coating. FIG. 3 illustrates that the surface coatings significantly decreased the surface temperature for a given voltage applied.

The drug loxapine was applied to a plain control foil and a foil having the 9 ml/cm² surface structure and allowed to dry. Loxapine has the following structure:

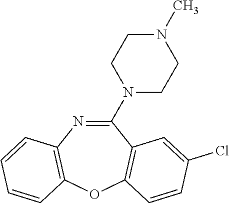 [SMILES: CN1CCN(C2=Nc3ccccc3Oc3ccc(Cl)cc32)CC1]

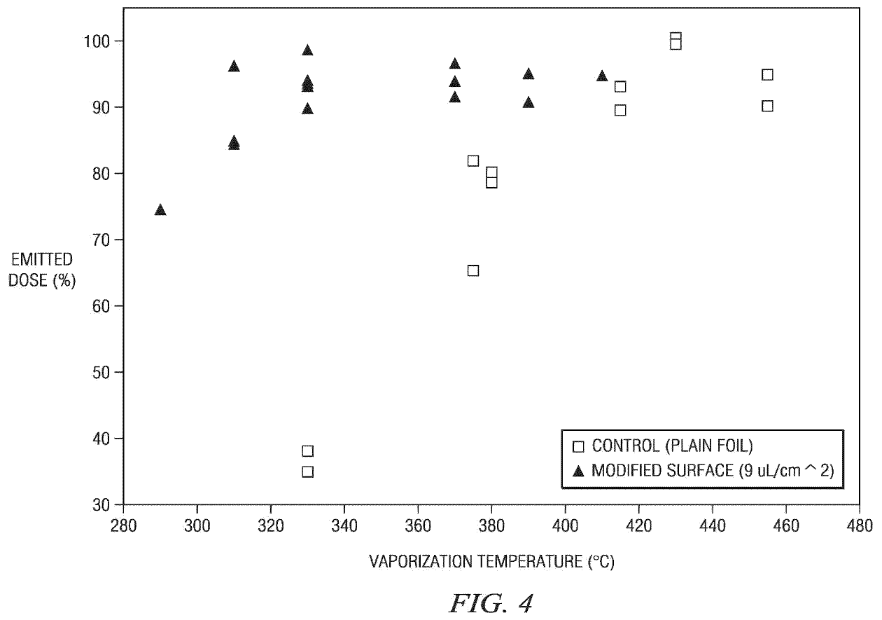

A loxapine coating was applied to both the surface structure and the plain foil which provided a 20 μm thick loxapine coating on the plain foil. The substrates were then heated to various temperatures and the emitted dose was measured. These results are illustrated in FIG. 4. As can be seen, the surface structure enabled release of a high percentage of the loxapine at relatively low temperatures as compared to the plain foil control.

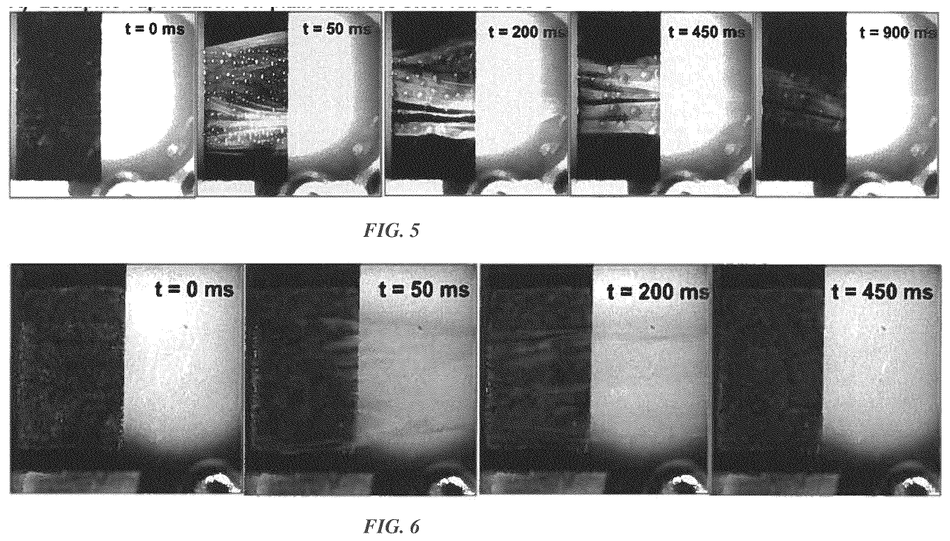

Finally, time lapse photographs were taken of the loxapine vaporization off of plain stainless steel foil 380° C. and off the surface structure of the example (9 ml/cm² of particle/binder slurry) at 380° C. (See FIGS. 5 and 6). Referring to FIG. 5, the loxapine on the plain stainless steel foil formed droplets of diameters as great as 2 mm (or higher) during vaporization. Referring to FIG. 6, the surface structure prevented formulation of visible drops of loxapine during vaporization. It is estimated that because the droplets were not visible, they were not of a size greater than about 10-20 μm.

The description of the present invention has been presented for purposes of illustration and description, but is not intended to be exhaustive or limiting of the invention to the form disclosed. The scope of the present invention is limited only by the scope of the following claims. Many modifications and variations will be apparent to those of ordinary skill in the art. The embodiment described and shown in the figures was chosen and described in order to best explain the principles of the invention, the practical application, and to enable others of ordinary skill in the art to understand the invention for various embodiments with various modifications as are suited to the particular use contemplated.

Various embodiments of the disclosure could also include permutations of the various elements recited in the claims as if each dependent claim was multiple dependent claim incorporating the limitations of each of the preceding dependent claims as well as the independent claims. Such permutations are expressly within the scope of this disclosure. All references cited herein are incorporated in their entirety by reference. As used herein; “or” means “and/or” unless otherwise specified.

What is claimed is:

1. A method of making a device for vaporizing a composition, comprising:
providing a thermally conductive substrate having a first surface;
forming a high surface area surface structure on the first surface;
applying a liquid drug composition to the high surface area structure; and
drying the liquid to leave a dry layer of the composition coating at least a portion of the high surface area structure.

2. The method of claim 2 wherein the high surface area structure is formed by applying a slurry of thermally conductive particles and an inorganic binder to the first surface of the thermally conductive substrate and drying the inorganic binder.

3. The method of claim 3 wherein the high surface area structure is formed by a process selected from a group comprising one or more of chemical etching, laser etching or cladding an area of the first surface.
